(19) United States
(12) Patent Application Publication
(10) Pub. No.: US 2024/0423751 A1
(43) Pub. Date: Dec. 26, 2024

(54) CONTROLLING A REPOSITIONABLE STRUCTURE SYSTEM BASED ON A GEOMETRIC RELATIONSHIP BETWEEN AN OPERATOR AND A COMPUTER-ASSISTED DEVICE

(71) Applicant: INTUITIVE SURGICAL OPERATIONS, INC., Sunnyvale, CA (US)

(72) Inventors: Mohammad Sina PARASTEGARI, Cupertino, CA (US); Jason A. GLASSER, San Francisco, CA (US); Olga GREENBERG, San Jose, CA (US); Paul G. GRIFFITHS, Santa Clara, CA (US); Katherine LI, San Jose, CA (US); Allen C. THOMPSON, Los Altos, CA (US); Keith J. WATZA, Boulder, CO (US)

(21) Appl. No.: 18/703,089

(22) PCT Filed: Oct. 21, 2022
(86) PCT No.: PCT/US2022/047480

§ 371 Date: Apr. 19, 2024

Related U.S. Application Data
(60) Provisional application No. 63/270,742, filed on Oct. 22, 2021.

(51) Int. Cl. A61B 90/00; A61B 34/35; A61B 90/60; G06F 3/01

(52) U.S. Cl. CPC A61B 90/37; A61B 34/35; A61B 90/60; G06F 3/012; G06F 3/013; A61B 2090/368; A61B 2090/372

(57) ABSTRACT

Techniques for controlling a computer-assisted device based on a geometric parameter representative of a geometric relationship of an operator and a display unit include the following. The computer-assisted device comprises a repositionable structure system configured to support a display unit that displays an image viewable by an operator; an actuator system physically coupled to the repositionable structure system; a sensor system configured to capture sensor data associated with a portion of a head of the operator; and a control system. The control system is configured to: determine, based on sensor data, a geometric parameter of the portion of the head relative to a portion of the computer-assisted device, determine a commanded motion based on the geometric parameter and a target parameter, and command the actuator system to move the repositionable structure based on the commanded motion.

### RELATED APPLICATIONS

This application claims the benefit to U.S. Provisional Application No. 63/270,742, filed Oct. 22, 2021, and entitled “Controlling A Repositionable Structure System Based On A Geometric Relationship Between An Operator And A Computer-Assisted Device,” which is incorporated by reference herein.

### TECHNICAL FIELD

The present disclosure relates generally to electronic devices and more particularly to controlling a repositionable structure based on a geometric relationship between an operator and a computer-assisted device.

### BACKGROUND

Computer-assisted electronic devices are being used more and more often. This is especially true in industrial, entertainment, educational, and other settings. As a medical example, the medical facilities of today have large arrays of electronic devices being found in operating rooms, interventional suites, intensive care wards, emergency rooms, and/or the like. Many of these electronic devices may be capable of autonomous or semi-autonomous motion. It is also known for personnel to control the motion and/or operation of electronic devices using one or more input devices located at a user control system. As a specific example, minimally invasive, robotic telesurgical systems permit surgeons to operate on patients from bedside or remote locations. Telesurgery refers generally to surgery performed using surgical systems where the surgeon uses some form of remote control, such as a servomechanism, to manipulate surgical instrument movements rather than directly holding and moving the instruments by hand.

When an electronic device is used to perform a task at a worksite, one or more imaging devices (e.g., an endoscope) can capture images of the worksite that provide visual feedback to an operator who is monitoring and/or performing the task. The imaging device(s) may be controllable to update a view of the worksite that is provided, such as via a display unit, to the operator. The display unit may have lenses and/or view screens.

To use the display unit, the operator positions his or her eyes so as to see images displayed on one or more view screens directly or through one or more intervening components. However, when the eyes are positioned at a less optimal position relative to the images, the operator may have a less optimal view of the images being displayed. Example effects of less optimal views of images include being unable to see an entire image being displayed, seeing stereoscopic images that do not properly fuse, etc. As a result, the operator may experience frustration, eye fatigue, inaccurate depictions of the items in the images, etc.

Accordingly, improved techniques for improving the positioning or orientation of the eyes of operators and images presented by display units are desirable.

### SUMMARY

Consistent with some embodiments, a computer-assisted device includes: a repositionable structure system, an actuator system, a sensor system, and a control system. The repositionable structure system is configured to physically couple to a display unit, and the display unit is configured to display images viewable by an operator. The actuator system is physically coupled to the repositionable structure system, and the actuator system is drivable to move the repositionable structure. The sensor system is configured to capture sensor data associated with a portion of a head of the operator. The control system is communicably coupled to the actuator system and the sensor system, and the control system is configured to: determine, based on the sensor data, a geometric parameter of the portion of the head relative to a portion of the computer-assisted device, determine a commanded motion based on the geometric parameter and a target parameter, and command the actuator system to move the repositionable structure system based on the commanded motion. The geometric parameter is representative of a geometric relationship of at least one eye of the operator relative to one or more images displayed by the display unit. The portion of the computer-assisted device, consistent with some embodiments, is selected from the group consisting of: portions of the display unit and portions of the repositionable structure system.

Consistent with some embodiments, a method includes determining, based on sensor data, a geometric parameter of a portion of a head of an operator relative to a portion of a computer-assisted device. The computer-assisted device comprises a repositionable structure system configured to physically couple to a display unit. The display unit is configured to display images. The geometric parameter is representative of a geometric relationship of at least one eye of the operator relative to the image(s) displayed by the display unit. The method further comprises determining a commanded motion based on the geometric parameter and a target parameter, and commanding an actuator system to move the repositionable structure system based on the commanded motion.

Other embodiments include, without limitation, one or more non-transitory machine-readable media including a plurality of machine-readable instructions, which when executed by one or more processors, are adapted to cause the one or more processors to perform any of the methods disclosed herein.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a simplified diagram including an example of a computer-assisted device, according to various embodiments.

FIG. 2 is a perspective view of an example display system, according to various embodiments.

FIG. 3 illustrates various approaches for controlling a repositionable structure system based on a geometric parameter, according to various embodiments.

FIG. 4 illustrates an approach for determining a geometric parameter between one or more portions of the head of an operator and one or more portions of a computer-assisted device, according to various embodiments.

FIG. 5 illustrates another approach for determining a geometric parameter between one or more portions of the head of an operator and one or more portions of a computer-assisted device, according to various embodiments.

FIG. 6 illustrates another approach for determining a geometric parameter between one or more portions of the head of an operator and one or more portions of a computer-assisted device, according to various embodiments.

FIG. 7 illustrates another approach for determining a geometric parameter between one or more portions of the head of an operator and one or more portions of a computer-assisted device, according to various embodiments.

FIG. 8 illustrates a simplified diagram of a method for adjusting a geometric parameter between one or more portions of the head of an operator and one or more portions of a computer-assisted device, according to various embodiments.

FIG. 9 illustrates a simplified diagram of a method for adjusting a repositionable structure system in response to entry into a mode in which a display unit is commanded to move based on head force and/or torque measurements, according to various embodiments.

### DETAILED DESCRIPTION

This description and the accompanying drawings that illustrate inventive aspects, embodiments, embodiments, or modules should not be taken as limiting-the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Further, the terminology in this description is not intended to limit the invention. For example, spatially relative terms-such as “beneath”, “below”, “lower”, “above”, “upper”, “proximal”, “distal”, and the like-may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, embodiment, or module may, whenever practical, be included in other embodiments, embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, embodiment, or application may be incorporated into other embodiments, embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various devices, elements, and portions of computer-assisted devices and elements in terms of their state in three-dimensional space. As used herein, the term “position” refers to the location of an element or a portion of an element in a three-dimensional space (e.g., three degrees of translational freedom along Cartesian x-, y-, and z-coordinates). As used herein, the term “orientation” refers to the rotational placement of an element or a portion of an element (three degrees of rotational freedom—e.g., roll, pitch, and yaw). As used herein, the term “shape” refers to a set positions or orientations measured along an element. As used herein, and for a device with repositionable arms, the term “proximal” refers to a direction toward the base of the computer-assisted device along its kinematic chain and “distal” refers to a direction away from the base along the kinematic chain.

Aspects of this disclosure are described in reference to computer-assisted systems and devices, which may include systems and devices that are teleoperated, remote-controlled, autonomous, semiautonomous, robotic, and/or the like. Further, aspects of this disclosure are described in terms of an embodiment using a medical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary, and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

### System Overview

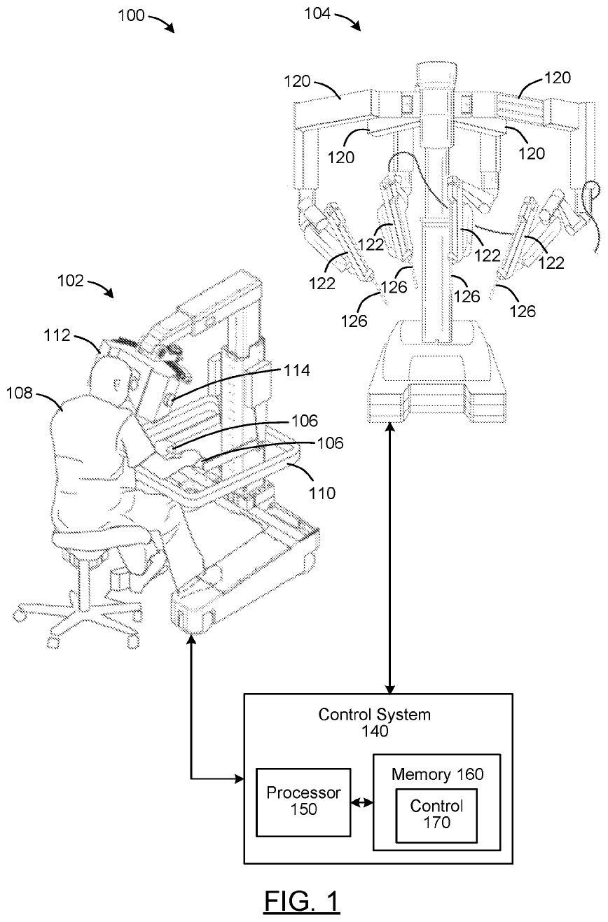

FIG. 1 is a simplified diagram of an example computer-assisted device, according to various embodiments. In some examples, the computer-assisted device is a teleoperated system 100. In medical examples, the teleoperated system 100 can be a teleoperated medical system such as a surgical system. As shown, the teleoperated system 100 includes a follower device 104. The follower device 104 is controlled by one or more leader input devices, described in greater detail below. Systems that include a leader device and a follower device are also sometimes referred to as master-slave systems. Also shown in Figure I is an input system that includes a workstation 102 (e.g., a console), and in various embodiments the input system can be in any appropriate form and may or may not include a workstation.

In this example, the workstation 102 includes one or more leader input devices 106 which are contacted and manipulated by an operator 108. For example, the workstation 102 can comprise one or more leader input devices 106 for use by the hands of the operator 108. The leader input devices 106 in this example are supported by the workstation 102 and can be mechanically grounded. In some embodiments, an ergonomic support 110 (e.g., forearm rest) can be provided on which the operator 108 can rest his or her forearms. In some examples, the operator 108 can perform tasks at a worksite near the follower device 104 during a procedure by commanding the follower device 104 using the leader input devices 106.

A display unit 112 is also included in the workstation 102. The display unit 112 can display images for viewing by the operator 108. The display unit 112 can be moved in various degrees of freedom to accommodate the viewing position of the operator 108 and/or to optionally provide control functions as another leader input device. In the example of the teleoperated system 100, displayed images can depict a worksite at which the operator 108 is performing various tasks by manipulating the leader input devices 106 and/or the display unit 112. In some examples, the images displayed by the display unit 112 can be received by the workstation 102 from one or more imaging devices arranged at the worksite. In other examples, the images displayed by the display unit 112 can be generated by the display unit 112 (or by a different connected device or system), such as for virtual representations of tools, the worksite, or for user interface components.

When using the workstation 102, the operator 108 can sit in a chair or other support in front of the workstation 102, position his or her eyes in front of the display unit 112, manipulate the leader input devices 106, and rest his or her forearms on the ergonomic support 110 as desired. In some embodiments, the operator 108 can stand at the workstation or assume other poses, and the display unit 112 and leader input devices 106 can be adjusted in position (height, depth, etc.) to accommodate the operator 108.

The teleoperated system 100 can also include the follower device 104, which can be commanded by the workstation 102. In a medical example, the follower device 104 can be located near an operating table (e.g., a table, bed, or other support) on which a patient can be positioned. In such cases, the worksite can be provided on the operating table, e.g., on or in a patient, simulated patient, or model, etc. (not shown). The teleoperated follower device 104 shown includes a plurality of manipulator arms 120, each configured to couple to an instrument assembly 122. An instrument assembly 122 can include, for example, an instrument 126 and an instrument carriage configured to hold a respective instrument 126.

In various embodiments, one or more of the instruments 126 can include an imaging device for capturing images (e.g., optical cameras, hyperspectral cameras, ultrasonic sensors, etc.). For example, one or more of the instruments 126 could be an endoscope assembly that includes an imaging device, which can provide captured images of a portion of the worksite to be displayed via the display unit 112.

In some embodiments, the follower manipulator arms 120 and/or instrument assemblies 122 can be controlled to move and articulate the instruments 126 in response to manipulation of leader input devices 106 by the operator 108, so that the operator 108 can perform tasks at the worksite. The manipulator arms 120 and instrument assemblies 122 are examples of repositionable structures on which instruments and/or imaging devices can be mounted. The repositionable structure(s) of a computer-assisted device comprise the repositionable structure system of the computer-assisted device. For a surgical example, the operator could direct the follower manipulator arms 120 to move instruments 126 to perform surgical procedures at internal surgical sites through minimally invasive apertures or natural orifices.

As shown, a control system 140 is provided external to the workstation 102 and communicates with the workstation 102. In other embodiments, the control system 140 can be provided in the workstation 102 or in the follower device 104. As the operator 108 moves leader input device(s) 106, sensed spatial information including sensed position and/or orientation information is provided to the control system 140 based on the movement of the leader input devices 106. The control system 140 can determine or provide control signals to the follower device 104 to control the movement of the manipulator arms 120, instrument assemblies 122, and/or instruments 126 based on the received information and operator input. In one embodiment, the control system 140 supports one or more wired communication protocols, (e.g., Ethernet, USB, and/or the like) and/or one or more wireless communication protocols (e.g., Bluetooth, IrDA, HomeRF, IEEE 1002.11, DECT, Wireless Telemetry, and/or the like).

The control system 140 can be implemented on one or more computing systems. One or more computing systems can be used to control the follower device 104. In addition, one or more computing systems can be used to control components of the workstation 102, such as movement of a display unit 112.

As shown, the control system 140 includes a processor 150 and a memory 160 storing a control module 170. In some embodiments, the control system 140 can include one or more processors, non-persistent storage (e.g., volatile memory, such as random access memory (RAM), cache memory), persistent storage (e.g., a hard disk, an optical drive such as a compact disk (CD) drive or digital versatile disk (DVD) drive, a flash memory, etc.), a communication interface (e.g., Bluetooth interface, infrared interface, network interface, optical interface, etc.), and numerous other elements and functionalities. In addition, functionality of the control module 170 can be implemented in any technically feasible software and/or hardware.

Each of the one or more processors of the control system 140 can be an integrated circuit for processing instructions. For example, the one or more processors can be one or more cores or micro-cores of a processor, a central processing unit (CPU), a microprocessor, a field-programmable gate array (FPGA), an application-specific integrated circuit (ASIC), a digital signal processor (DSP), a graphics processing unit (GPU), a tensor processing unit (TPU), and/or the like. The control system 140 can also include one or more input devices, such as a touchscreen, keyboard, mouse, microphone, touchpad, electronic pen, or any other type of input device.

A communication interface of the control system 140 can include an integrated circuit for connecting the computing system to a network (not shown) (e.g., a local area network (LAN), a wide area network (WAN) such as the Internet, mobile network, or any other type of network) and/or to another device, such as another computing system.

Further, the control system 140 can include one or more output devices, such as a display device (e.g., a liquid crystal display (LCD), a plasma display, touchscreen, organic LED display (OLED), projector, or other display device), a printer, a speaker, external storage, or any other output device. One or more of the output devices can be the same or different from the input device(s). Many different types of computing systems exist, and the aforementioned input and output device(s) can take other forms.

In some embodiments, the control system 140 can be connected to or be a part of a network. The network can include multiple nodes. The control system 140 can be implemented on one node or on a group of nodes. By way of example, the control system 140 can be implemented on a node of a distributed system that is connected to other nodes. By way of another example, the control system 140 can be implemented on a distributed computing system having multiple nodes, where different functions and/or components of the control system 140 can be located on a different node within the distributed computing system. Further, one or more elements of the aforementioned control system 140 can be located at a remote location and connected to the other elements over a network.

Software instructions in the form of computer readable program code to perform embodiments of the disclosure can be stored, in whole or in part, temporarily or permanently, on a non-transitory computer readable medium such as a CD, DVD, storage device, a diskette, a tape, flash memory, physical memory, or any other computer readable storage medium. Specifically, the software instructions can correspond to computer readable program code that, when executed by a processor(s) (e.g., processor 150), is configured to perform some embodiments of the methods described herein.

In some embodiments, the one or more leader input devices 106 can be ungrounded (ungrounded leader input devices being not kinematically grounded, such as leader input devices held by the hands of the operator 108 without additional physical support). Such ungrounded leader input devices can be used in conjunction with the display unit 112. In some embodiments, the operator 108 can use a display unit 112 positioned near the worksite, such that the operator 108 manually operates instruments at the worksite, such as a laparoscopic instrument in a surgical example, while viewing images displayed by the display unit 112.

Some embodiments can include one or more components of a teleoperated medical system such as a da Vinci® Surgical System, commercialized by Intuitive Surgical, Inc. of Sunnyvale, California, U.S.A. Embodiments on da Vinci® Surgical Systems are merely examples and are not to be considered as limiting the scope of the features disclosed herein. For example, different types of teleoperated systems having follower devices at worksites, as well as non-teleoperated systems, can make use of features described herein.

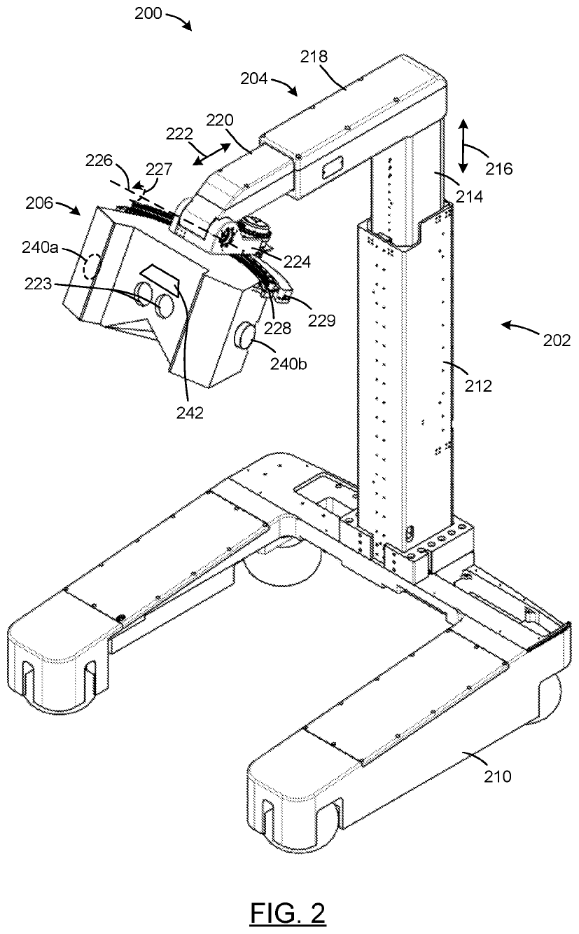

FIG. 2 is a perspective view of an example display system 200 of a computer-assisted device, according to various embodiments. In some embodiments, the display system 200 is used in a workstation of a teleoperated system (e.g., in workstation 102 of the teleoperated system 100 of FIG. 1), or the display system 200 can be used in other systems or as a standalone system, e.g., to allow an operator to view a worksite or other physical site, a displayed virtual environment, etc. Although FIGS. 2-7 show specific configurations of the display system 200, other embodiments may use different configurations.

As shown in FIG. 2, the display system 200 includes a base support 202, an arm support 204, and a display unit 206. The display unit 206 is provided with multiple degrees of freedom of movement provided by a support linkage including base support 202, an arm support 204 coupled to the base support 202, and a tilt member 224 (described below) coupled to the arm support 204, where the display unit 206 is coupled to the tilt member 224.

The base support 202 can be a vertical member that is mechanically grounded, e.g., directly or indirectly coupled to ground, such as by resting or being attached to a floor. For example, the base support 202 can be mechanically coupled to a wheeled support structure 210 that is coupled to the ground. The base support 202 includes a first base portion 212 and a second base portion 214 coupled such that the second base portion 214 is translatable with respect to the first base portion 212 in a linear degree of freedom 216.

The arm support 204 can be a horizontal member that is mechanically coupled to the base support 202. The arm support 204 includes a first arm portion 218 and a second arm portion 220. The second arm portion 220 is coupled to the first arm portion 218 such that the second arm portion 220 is linearly translatable in a first linear degree of freedom (DOF) 222 with respect to the first arm portion 218.

The display unit 206 can be mechanically coupled to the arm support 204. The display unit 206 can be moveable in other linear DOFs provided by the linear translations of the second base portion 214 and the second arm portion 220.

In some embodiments, the display unit 206 includes a display, e.g., one or more display screens, projectors, or the like that can display digitized images. In the example shown, the display unit 206 further includes lenses 223 that provide viewports through which the display device can be viewed. As used herein, “lenses” refers to a single lens or multiple lenses, such as a separate lens for each eye of an operator, and “eyes” refers to a single eye or both eyes of an operator. Any technically feasible lenses can be used in embodiments, such as lenses having high optical power. Although display units that include lenses, through which images are viewed, are described herein as a reference example, some embodiments of display units may not include such lenses. For example, in some embodiments, the images displayed by a display unit can be viewed via an opening that allows the viewing of displayed images, viewed directly as displayed by a display screen of the display unit, or in any other technically feasible manner.

In some embodiments, the display unit 206 displays images of a worksite (e.g., an interior anatomy of a patient in a medical example), captured by an imaging device such as an endoscope. The images can alternatively depict a virtual representation of a worksite that are computer-generated. The images can show captured images or virtual renderings of instruments 126 of the follower device 104 while one or more of these instruments 126 are controlled by the operator via the leader input devices (e.g., the leader input devices 106 and/or the display unit 206) of the workstation 102.

In some embodiments, the display unit 206 is rotationally coupled to the arm support 204 by a tilt member 224. In the illustrated example, the tilt member 224 is coupled at a first end to the second arm portion 220 of the arm support 204 by a rotary coupling configured to provide rotational motion of the tilt member 224 and the display unit 206 about the tilt axis 226 with respect to the second arm portion 220.

Each of the various degrees of freedom discussed herein can be passive and require manual manipulation for movement, or be movable by one or more actuators, such as by one or more motors, solenoids, etc. For example, the rotational motion of the tilt member 224 and the display unit 206 about the tilt axis 226 can be driven by one or more actuators, such as by a motor coupled to the tilt member at or near the tilt axis 226.

The display unit 206 can be rotationally coupled to the tilt member 224 and can rotate about a yaw axis 230. For example, the rotation can be a lateral or left-right rotation from the point of view of an operator viewing images displayed by the display unit 206. In this example, the display unit 206 is coupled to the tilt member by a rotary mechanism which can comprise a track mechanism that constrains the motion of the display unit 206. For example, in some embodiments, the track mechanism includes a curved member 228 that slidably engages a track 229, thus allowing the display unit 206 to rotate about a yaw axis by moving the curved member 228 along the track 229.

The display system 200 can thus provide the display unit 206 with a vertical linear degree of freedom 216, a horizontal linear degree of freedom 222, and a rotational (tilt) degree of freedom 227. A combination of coordinated movement of components of the display system 200 in these degrees of freedom allows the display unit 206 to be positioned at various positions and orientations based on the preferences of an operator. The motion of the display unit 206 in the tilt, horizontal, and vertical degrees of freedom allow the display unit 206 to stay close to, or maintain contact with, the head of the operator, such as when the operator is providing head input through head motion when the display system 200 is in a head input mode.

In the head input mode, the control system of the computer-assisted device commands the repositionable structure system to move the display unit 206 based on at least one head input selected from the group consisting of: a head motion, an applied force of the head, and an applied torque of the head. The head input may be acquired via a sensor, such as a pressure sensor disposed on a surface of the headrest 242, a force and/or torque sensor embedded in the headrest 242 or disposed in a force/torque transmitting support of the headrest 242, a sensor located in a repositionable structure coupled to the headrest 242, etc. Thus, in some embodiments, the operator can move his or her head to provide input to control the display unit 206 to move with the head such that it appears to “follow” the motion of the head. In various embodiments, the movement of the display unit 206 in head input mode can be for ergonomic adjustments, to enable the operator to use the display unit 206 as an input device for commanding teleoperation of a manipulator arm, etc.

In various embodiments, the control system is configured with no head input mode, with a single head input mode, or with a plurality of different head input modes (e.g., a first head input mode for ergonomic adjustments, a second head input mode for teleoperation, etc.). In some embodiments, motions of the head in a head input mode can be used to provide teleoperative control of the position and/or orientation of imaging devices that capture images displayed via the display unit 206 and/or other devices. For example, the control system can be configured to use measurements of the forces and/or torques applied by the head, motion of the display unit 206, or motion of a repositionable structure coupled to the display unit 206, to determine teleoperation commands for such teleoperative control. Thus, in various embodiments that support head input mode(s), the control system can be configured such that motion of the display unit 206 is not associated with providing commands for the teleoperative control in the head input mode, is associated with providing commands for teleoperative control in the head input mode, or is associated with providing commands for teleoperative control in a first mode and not in a second mode. In various embodiments supporting head input modes, the control system may also be configured with one or more other modes, such as a mode in which the display unit 206 cannot be commanded to move by head input, or cannot be commanded to move at all. Further, in some embodiments, the display unit 206 is supported by a structure that is not repositionable, i.e., cannot be physically moved by the actuator system.

In embodiments with and without head input modes, and while operating in a head input mode, the position and/or orientation of one or more instruments (including instruments comprising imaging devices that capture images displayed via the display unit 206) can be controlled using devices other than the display unit 206, such as via the leader input devices 106 that are manipulated by the hands of an operator.

Illustratively, the display unit 206 is coupled to a headrest 242. The headrest 242 can be separate from, or integrated within the display unit 206, in various embodiments. In some embodiments, the headrest 242 is coupled to a surface of the display unit 206 that is facing the head of the operator during operation of the display unit 206. The headrest 242 is configured to be able to contact the head of the operator, such as a forehead of the operator. In some embodiments, the headrest 242 can include a head-input sensor that senses inputs applied to the headrest 242 or the display unit 206 in a region above the lenses 223. The head-input sensor can include any of a variety of types of sensors, e.g., resistance sensors, capacitive sensors, force sensors, optical sensors, etc. In some embodiments, the head-input sensor is configured to be in contact with the forehead of the operator while the operator is viewing images. In some embodiments, the headrest 242 is static and does not move relative to a housing of the display unit 206. In some embodiments, the headrest 242 is physically coupled to the repositionable structure system. That is, the headrest 242 is physically coupled to at least one repositionable structure of the repositionable structure system; where the repositionable structure system comprises multiple repositionable structures, the headrest 242 may be coupled to the repositionable structure system by being coupled to only one of the multiple repositionable structures.

For example, the headrest 242 may be mounted on or otherwise physically coupled to a repositionable structure (e.g., linkage, a linear slide, and/or the like) and can be moved relative to the housing of the display unit 206 by movement of the repositionable structure. In some embodiments, the repositionable structure may be moved by reconfiguration through manual manipulation and/or driving of one or more actuators of the actuator system of the computer-assisted device. The display unit 206 can include one or more head input sensors that sense operator head input as commands to cause movement of the imaging device, or otherwise cause updating of the view in the images presented to the operator (such as by graphical rendering, digital zooming or panning, etc.). Further, in some embodiments and some instances of operation, the sensed head movement is used to move the display unit 206 to compensate for the head movement. The position of the head of the operator can, thus, remain stationary relative to at least part of the display unit 206, such as to the lenses 223, even when the operator performs head movements to control the view provided by the imaging device.

It is understood that FIG. 2 merely shows an example for a configuration of a display system. Alternative configurations supporting movement of the display unit 206 based on an input from the operator are also possible. Any repositionable structure that supports the display unit 206 and provides it with degrees of freedom and ranges of motion appropriate for the application can be used in lieu of the configuration shown in FIG. 2. Additional examples of moveable display systems are described in International Patent Application Publication No. WO 2021/041249 and entitled “Moveable Display System,” which is incorporated by reference herein.

Although described herein primarily with respect to the display unit 206 that is part of a grounded mechanical structure (e.g., the display system 200), in other embodiments, the display unit can be any technically feasible display device or devices. In all of these cases, the position and/or orientation of the display unit can be determined using one or more accelerometers, gyroscopes, inertial measurement units, cameras, and/or other sensors internal or external to the display unit.

### Commanding a Repositionable Structure Based on a Geometric Parameter Between One or More Portions of an Operator and One or More Portions of a Computer-Assisted Device

The display unit (or lenses of the display unit or a headrest if the display unit has lenses or is coupled to a headrest) can be adjusted to reposition a geometric relationship of the eye(s) of an operator relative to image(s) displayed by the display unit based on a target geometric parameter.

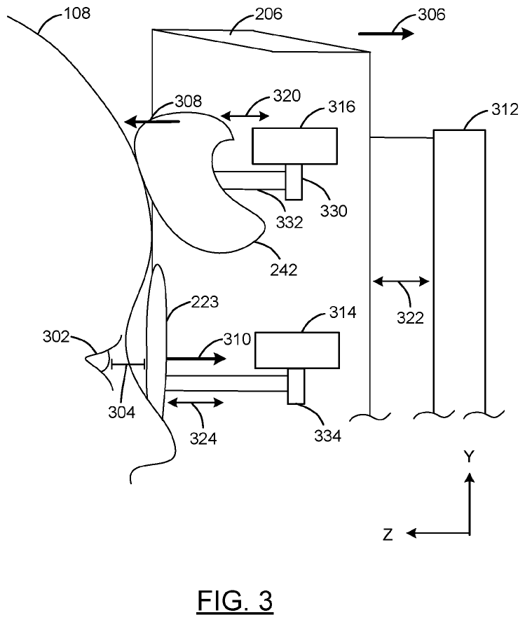

FIG. 3 illustrates various approaches for controlling a repositionable structure of a repositionable structure system based on a geometric parameter between one or more portions of the head of an operator and one or more portions of a computer-assisted device, according to various embodiments. In FIG. 3, the one or more portions of the head comprise eye(s) of the operator, and the one or more portions of the computer-assisted device comprise one or more portions of the display system 200 (e.g., which can be one or more portions of the display unit 206 of the display system 200).

As shown, a geometric parameter is determined using sensor data. The geometric parameter is representative of a geometric relationship between one or more eyes (e.g., eye 302) of the operator 108 and an image displayed by the display system 200; for example, the geometric relationship may be an optical distance from one or more eyes (e.g., eye 302) of the operator 108 and an image displayed by the display system 200. The geometric parameter is representative of the geometric relationship in that a static transformation or determinable transformation exists between the geometric parameter and the geometric relationship. For example, a geometric parameter comprising a distance between one or more eyes of the operator to a feature of a housing of the display unit may be used with information about relative geometries between that feature and other display unit components, and optical characteristics of optical elements of the display unit, to represent a geometric relationship of an optical distance between the one or more eyes to image(s) shown by the display unit. The relative geometries may be known from the physical design of the display unit, calibration measurements, sensors configured to detect the configuration of the display unit, and the like. As another example, a geometric parameter comprising a relative location between a nose of the operator to a link of a repositionable structure of a repositionable structure system physically coupled to the display unit can be used to represent a geometric relationship of an optical offset between the one or more eyes to image(s) shown by the display unit; the location of the operator's eyes can be determined from the location of the nose. Kinematic information of the repositionable structure obtained from sensors or pre-programmed information (e.g., regarding lengths of links, etc.) can be used to locate the display unit relative to the nose or eyes. Then, similar information about the display unit as above can be used to associate the geometric parameter with the geometric relationship. As noted above, the geometric parameter may be used as-is to determine commanded motion, or can be used to provide intermediate or final calculations of the geometric relationship in determining commanded motion.

As a specific example, the geometric parameter can comprise a distance 304 from the eye(s) (e.g., eye 302) of the operator 108 to one or more portions of the display system 200. For illustration, the following examples are discussed herein primarily with the distance 304 being that from the eye(s) (e.g., eye 302) of an operator to one or more lenses (e.g., lenses 223) of a display unit (e.g., display unit 206). The distance from the eye(s) to the lens(es) are also referred to herein as the eye-to-lenses distance; in these examples, each lens of the one or more lenses are positioned between a location of images being displayed and an expected location of at least one eye. Thus, the eye-to-lenses distance is used as a reference example in much of the discussion herein. In various embodiments, any technically feasible geometric parameter that is representative of a geometric relationship of the eye(s) of an operator relative to the image(s) displayed by display unit can be determined. The images can be viewed as displayed on a display screen, from a lens or other optical element that is between the display screen and the eyes, or in any other technically feasible manner. The geometric relationship may or may not be calculated for a commanded motion, and commanded motions can be based on the geometric parameter as determined, or be based on the geometric parameter through the use of information derived using the geometric parameter (e g., the geometric relationship, if the geometric relationship is calculated).

In some embodiments, the geometric parameter is of a portion of the head relative to a portion of the computer-assisted device, where the portion of the computer assisted device is selected from the group consisting of: portions of the display unit and portions of the repositionable structure system. In some embodiments, the geometric parameter comprises a distance from the portion of the head to the portion of the computer-assisted device. As some examples, the geometric parameter can be a distance from portion(s) of the head of an operator to portion(s) of a display unit, a distance from portion(s) of the head to portion(s) of a repositionable structure system physically coupled to the display unit, a location of portion(s) of the head relative to portion(s) of the display unit, a location of portion(s) of the head relative to portion(s) of the repositionable structure system, and/or the like. In some embodiments, the geometric parameter can be a distance from at least one eye of an operator to a lens of a display unit, a distance from at least one eye to a part of the display unit other than a lens, a distance from at least one eye to image(s) displayed by the display unit, and/or the like. In various embodiments, the distance referred to previously may be a scaled or unscaled separation distance. In some embodiments, the distance, or another geometric parameter representative of a geometric relationship of the eye(s) of the operator 108 relative to the image(s) displayed by the display unit, such as one of the geometric parameters described above, can be determined in any technically feasible manner.

The display unit is physically coupled to the repositionable structure system by being physically coupled to at least one repositionable structure of the repositionable structure system. Thus, if the repositionable structure system comprises multiple repositionable structures, not all of the multiple repositionable structures need to be physically coupled to the display unit.

In some embodiments, the one or more portions of the head comprises at least one eye. In some embodiments, the one or more portions of the computer assisted device (e.g., display system 200) comprises a portion selected from the group consisting of: portions of a display unit (e.g., display unit 206) and portions of the repositionable structure system configured to physically couple to the display unit. In some embodiments, the one or more portions of the computer assisted device comprises a portion selected from the group consisting of: lenses of the display unit, a housing of the display unit, a display screen surface of the display unit, and links of the repositionable structure system.

The lenses 223, or other portion(s) of the display system 200, can then be repositioned based on a target parameter, such as a target distance (e.g., 15-20 mm) or a target location, relative to the eyes 302 of the operator 108, or other portion(s) of the head of the operator 108. In this example, moving the display unit 206 in accordance with the commanded motion determined based on the target parameter moves the display unit 206 relative to the eyes 302 so that the eyes 302 and the images displayed by the display unit 206 have an updated geometric relationship that can be represented by an updated geometric parameter, where the updated geometric parameter differs from the target parameter by less than the previous geometric parameter differed from the target parameter. Thus, moving the repositionable structure system coupled to the display unit 206 based on the commanded motion would cause the at least one eye to have an updated geometric relationship relative to the image; the updated geometric relationship is representable by the updated geometric parameter that differs from the target parameter by less than the (original) geometric parameter.

The target parameter is a geometric parameter that is similar in format to the geometric parameter described above. However, the target parameter is associated with a target for the geometric relationship represented by the geometric parameter that is measured or otherwise determined during operation of the display system 200. For example, the target parameter could set based on a distance from the lenses 223 to a focal point (not shown) associated with the lenses 223 or a distance from the lenses 223 to a viewing zone (not shown) within which eyes 302 of the operator 108 can perceive with acceptable focus and accuracy any information displayed by the display unit 206 through the lenses 223. Repositioning the lenses 223 or other portion(s) of the display system 200 based on the target parameter can improve the operator 108's view of images being displayed by display unit 206, such as increasing the ability of the operator 108 to see an entire image being displayed via the display unit 206 and/or to see a properly fused image that combines images seen by different eyes. The target parameter can be defined in part based on the type of lenses included in a display unit, one or more display related characteristics of the display unit (e.g., whether the display unit includes lenses, the display technology used, and/or the like), a physical configuration of the display unit (e.g., locations of the lenses relative to a display screen or optical element of the display unit), a calibration procedure, and/or operator preference, among other things.

In some embodiments, the target parameter can be set to a distance of the eyes 302 (or other portion(s) of the head of the operator 108) from portion(s) of the display system 200, such as the lenses 223, or a location of the portion(s) of the display system 200 relative to the eyes 302, at the completion of a manual adjustment to the position of the display unit 206 by the operator 108. For example, the operator 108 could press buttons, operate a finger switch, or otherwise cause the display unit 206 to be moved so that the operator 108 can view displayed images comfortably. These operator 108 adjustments can be part of a calibration procedure, and the target parameter can be set to the distance from the eyes 302 (or other portion(s) of the head of the operator 108) to the portion(s) of the display system 200 (e.g., the eye-to-lenses distance), or the location of the portion(s) of the display system relative to the eyes 302 (or other portion(s) of the head of the operator 108) at the completion of the adjustments.

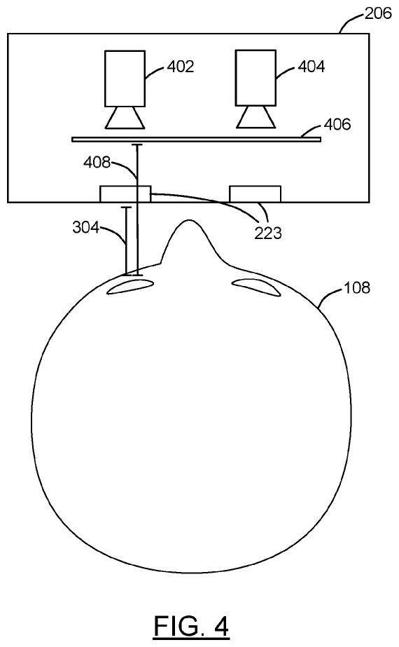

In some examples, a camera or other imaging device can be placed behind each lens 223, or elsewhere, to captures images of one or both eyes 302 of the operator 108. FIG. 4 illustrates an example configuration of the display system 200 in which cameras 402 and 404 are placed behind each of the lenses 223 and one or more optical elements. According to various embodiments, each camera 402, 404 is placed behind an optical element comprising a half-silvered mirror 406 that helps to conceal the cameras 402 and 404 from the view of the operator 108. Relative to the half-silvered mirror 406, the cameras 402, 404 are then placed in a direction away from the operator 108. Display images can be projected onto the half-silvered mirror 406 in some embodiments. Cameras 402, 404 or other imaging devices can be placed elsewhere in other embodiments. For example, cameras could be placed at dark locations within the display unit 206 that are not easily visible to the operator 108. As another example, fiber optics (e.g., the fiber optics in a fiber optic camera), lenses, and/or mirrors could be used to direct a view to one or more cameras that are located elsewhere. As a further example, the half-silvered mirror 406 can be replaced with other optical element(s), and cameras could be placed behind the other optical element(s).

As an example, in operation, the distance 304 between the eyes 302 of the operator 108 and the lenses 223, or another geometric parameter as described above (e.g., a distance 408 between the eyes 302 and images displayed on the half-silvered mirror 406), can be determined by estimating a distance between pupils of the operator 108 (also referred to herein as an “interpupillary distance”) in images that are captured by the cameras 402 and 404 (or other cameras or imaging devices) and comparing the estimated distance to a reference distance between the pupils of the operator 108. It should be understood that the distance between the pupils in the captured images will decrease relative to the reference distance between the pupils when the eyes 302 of the operator 108 move away from the lenses 223 and other portion(s) of the display system 200, such as when the operator 108 moves and/or tilts his or her head away, and vice versa. The pupils can be detected in the captured images using machine learning and/or any other computer vision techniques. The estimated distance can be determined in any technically feasible manner, including by converting distances in pixels of the images to real-world distances. The reference distance between the pupils can be obtained in any technically feasible manner, such as using a commercially-available device that measures the interpupillary distance of the operator 108 that is then stored in a profile for the operator, using a graphical user interface that permits the operator 108 to input his or her interpupillary distance, and/or by using a default distance when the interpupillary distance of a particular operator has not been measured. For example, the default distance could be between 62-65 mm. The distance 304, or another geometric parameter as described above, can then be calculated by inputting the estimated distance into a function (e.g., a linear function, a non-linear function) or a lookup table or other construct that relates a ratio between the estimated distance and the reference distance to the distance 304 or the other geometric parameter. The function, lookup table, or other construct can be obtained in any technically feasible manner, such as through extrapolation or interpolation of data, including through linear regression.

As another example, the distance 304, or another geometric parameter, can be determined by comparing the size of an iris or other immutable feature of the eyes 302 that is detected in the captured images with a reference size of the iris or other immutable feature using a similar function (e.g., a linear function, a non-linear function) or a lookup table or other construct. For example, the size could be a diameter of the iris. Similar to the reference inter-pupillary distance, the reference size of the iris or other immutable feature can be a measured size, a user-input size, or a default size (e.g., an average iris diameter) in some embodiments. In other embodiments, when the inter-pupillary distance or the size of the iris or other immutable feature is not known for a particular operator, no adjustments are made to the display unit 206, the lenses 223, or the headrest 242 to reposition the lenses 223 relative to the eyes 302 of the operator 108. In yet further embodiments, when the inter-pupillary distance or the size of the iris or other immutable feature is not known for the operator 108, adjustments can be made to the display unit 206, the lenses 223, or the headrest 242 to reposition the lenses 223 or other portion(s) of the display system 200 at a default target distance relative to the eyes 302 of the operator 108.

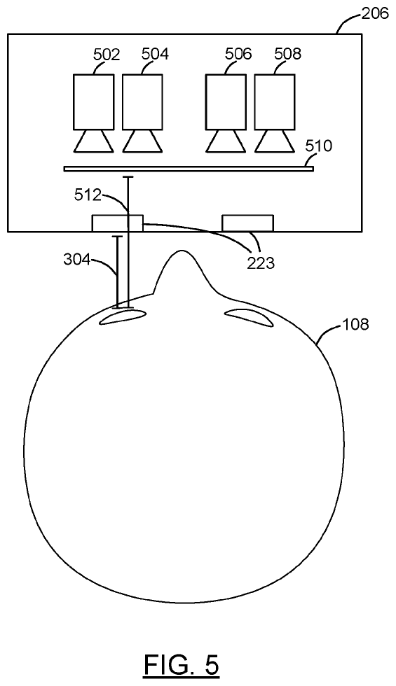

In some examples, a pair of cameras or other imaging devices can be placed behind each lens 223, or elsewhere, to capture stereo images of one or both eyes 302 of the operator 108. FIG. 5 illustrates an example configuration of the display system 200 in which pairs of cameras 502-504 and 506-508 are placed behind each of the lenses 223 and a half-silvered mirror 510 is used to conceal the cameras 502, 504, 506, and 508 from the operator 108, according to various embodiments. Similar to the description above in conjunction with FIG. 4, display images can be projected onto the half-silvered mirror 510 in some embodiments. Pairs of cameras can be placed elsewhere in other embodiments. Similar to the description above in conjunction with FIG. 4, in some embodiments, cameras can be placed at dark locations within the display unit 206, cameras can be placed behind optical elements other than half-silvered mirrors, or fiber optics (e.g., the fiber optics in a fiber optic camera), lenses, and/or mirrors can be used to direct a view to one or more cameras that are positioned elsewhere. In operation, the distance between each eye 302 of the operator 108 and a corresponding lens 223, or another geometric parameter as described above (e.g., a distance 408 between an eye 302 and images displayed on the half-silvered mirror 510), can be determined based on parallax between pupils that are detected in the stereo images via machine learning and/or any other computer vision techniques. When the distances between each eye 302 of the operator 108 and the corresponding lens 223 or other portions of the display system 200, or the other geometric parameter for each eye, are determined to be different, the different distances (or other geometric parameters) can be aggregated (e.g., averaged) to determine the distance 304 or an aggregated other geometric parameter.

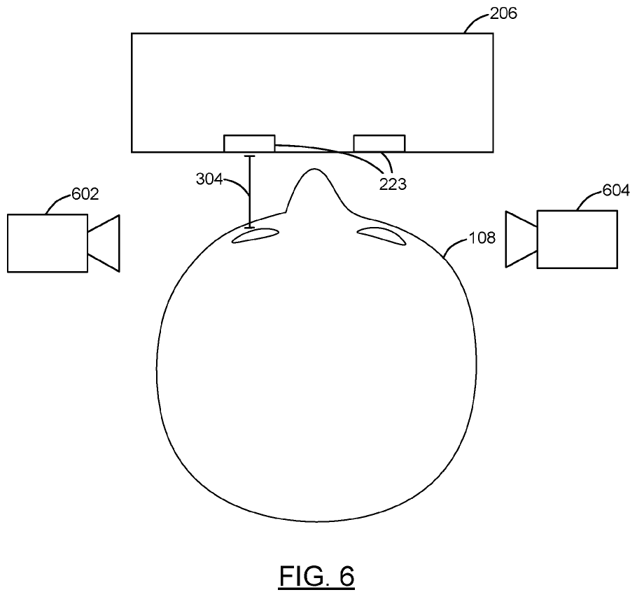

In some examples, one or more cameras can be positioned to capture one or more different views of the operator 108. FIG. 6 illustrates an example configuration of the display system 200 in which cameras 602 and 604 are used to capture images of two views of the operator 108, according to various embodiments. Although two cameras 602 and 604 are shown for illustrative purposes, in some embodiments, a display system can include one or a set of cameras that are positioned to capture any number of views of the operator 108. For example, some embodiments can include a single camera that captures one view of the operator 108. In operation, a distance from each eye 302 of the operator 108 to a corresponding lens 223, or another geometric parameter as described above (e.g., a distance between each eye 302 and images display on a display screen (not shown) that is in images captured by the cameras 602 and 604), can be determined by scaling a distance between that eye 302, or other portion(s) of the head of the operator 108, and the corresponding lens 223, or other portion(s) of the display system, in an image captured by the camera 602 or 604. Eyes 302 of the operator 108 and the lenses 223, or other portion(s) of operator 108 and of the display system 200, can be detected in images captured by the cameras 602 and 604 using machine learning or any other computer vision techniques. Assuming that the depth of the operator 108 (i.e., the distance from a viewer) within the captured images is known, the pixel distance between an eye 302 and a corresponding lens 223, or between other portion(s) of the head of the operator 108 and portion(s) of the display system 200, in an image can be scaled by a scaling factor that is proportional to the depth to determine an actual distance between that eye 302 and the corresponding lens 223, or between the other portion(s) of the head of the operator 108 and the other portion(s) of the display system 200.

As another example that can be used only when the eyes 302 or other portion(s) of the head of the operator 108 (and not the lenses 223 or other portion(s) of the display system 200) are captured in the images, the following technique can be performed. The distance between the eye 302 and a corresponding lens 223, or other geometric parameter, can be determined based on a position of the eye 302 or other portion(s) of the head of the operator 108 in one of the captured images and a reference position of the corresponding lens 223 or other portion(s) of the display system 200. When the distances between each eye 302 or portion of the head of the operator 108 and the corresponding lens 223 or other portion(s) of the display system 200 are determined to be different, the different distances can be aggregated (e.g., averaged) to determine the distance 304 or other geometric parameter. Alternatively, in some embodiments, a single distance between one eye 302 or other portion of the head of the operator 108 and a corresponding lens 223, or other portion(s) of the display system 200, can be determined, and the single distance can be used as the distance 304 or other geometric parameter.

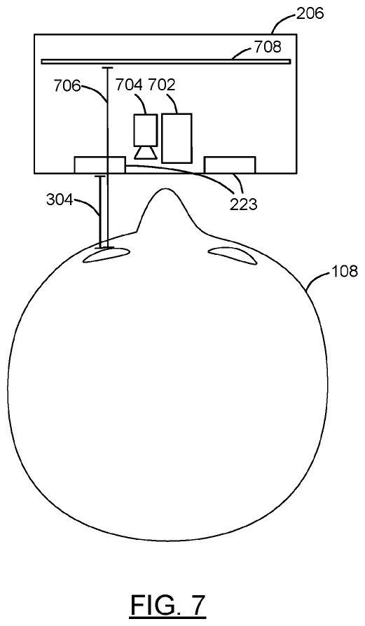

In some examples, a time-of-flight sensor, or other sensor device, can be used to measure distances to points on a face of the operator 108. FIG. 7 illustrates an example in which the display system 200 includes a time-of-flight sensor 702 and a camera 704 within the display unit 206, according to various embodiments. The time-of-flight sensor 702 can be any technically feasible sensor that emits signals and measures the return times of those signals to the time-of-flight sensor 702. The return times can then be converted to distance measurements. For example, the time-of-flight sensor 702 can be a LiDAR (Light Detection and Ranging) sensor in some embodiments. Other sensor devices that can be used to measure distance in some embodiments include an accelerometer or inertial sensor coupled directly or indirectly to the head, a camera, an emitter-receiver system with the emitter or received coupled directly or indirectly to the head, or a combination thereof. In operation, the distance 304, or another geometric parameter as described above (e.g., a distance 706 between the eyes 302 of the operator and images displayed on a display screen 708), can be determined by detecting the eyes 302 or other portion(s) of the head of the operator 108 in images captured by the camera 704 (or another imaging device) and computing the distance 304, or the distance from other portion(s) of the head of the operation to portion(s) of the display system 200, based on time-of-flight measurement data (or other sensor data) corresponding to the eyes 302 or other portion(s) of the head. The eyes 302 or other portion(s) of the head can be detected using machine learning and/or any other computer vision techniques, and corresponding points in the time-of flight measurement data can be detected or otherwise used to determine the distance between each eye 302 and a corresponding lens 223, or the distance from the other portion(s) of the head of the operator 108 to other portion(s) of the display system 200.

Further, the distances, or another geometric parameter computed for each eye 302 or portion of the head of the operator 108 can be averaged to determine the distance 304, or an aggregated other parameter, when the distances or other parameters are different for different eyes 302. Alternatively, in some embodiments, a single distance or another geometric parameter between one eye 302 or other portion of the head of the operator 108 and a corresponding lens 223 or other portion(s) of the display system 200 can be determined, and the single distance can be used as the distance 304, or another geometric parameter.

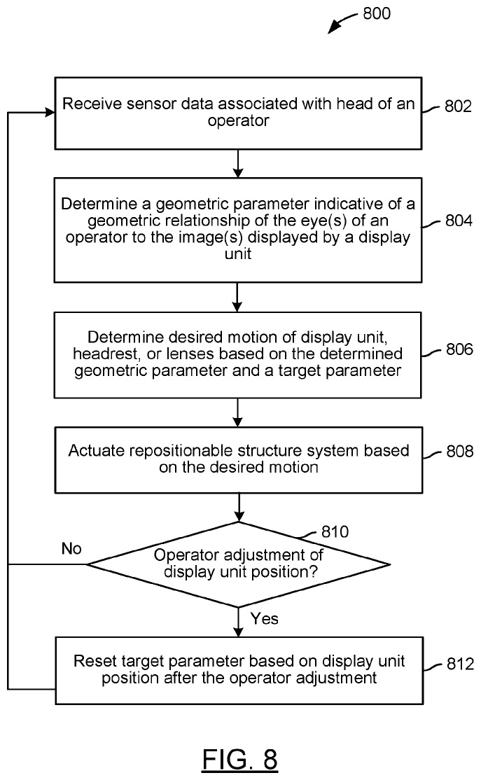

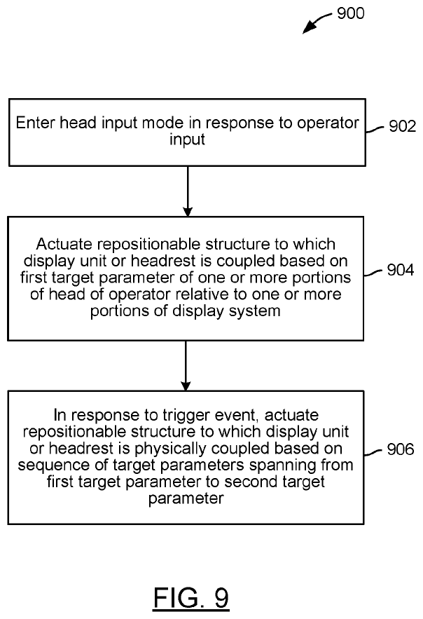

It should be noted that the distances measured by cameras on the sides of the operator 108 and by a time-to-flight sensor, described above in conjunction with FIGS. 8-9, are physical distances. By contrast, the distances measured by cameras and pairs of cameras that are pointed at the eyes 302 of the operator 108, described above in conjunction with FIGS. 6-7, are optical distances which can change depending on, e.g., whether the operator 108 is wearing glasses. Experience has shown that repositioning lenses relative to the eyes 302 of the operator 108 based on optical distances is more accurate than doing so based on physical distances.

Returning to FIG. 3, after the distance 304, or another geometric parameter as described above, is determined, the lenses 223 or other portion(s) of the display system 200 can be repositioned relative to the eyes 302 or other portion(s) of the head of the operator 108 based on the target parameter in any technically feasible manner. In some examples, the control module 170 can issue commands to a controller for actuators of joints of a repositionable structure system to which the display unit 206 is physically coupled, to cause movement of the display unit 206 in the “7” direction that is parallel to a direction of view of the operator 108. An example movement 306 of the display unit 206 in the Z direction (i.e., inward-outward) that increases the distance 304, or another geometric parameter, is shown in FIG. 3. As shown, the display unit 206 can generally be moved in a DOF 322 closer or farther away from the eyes 302 of the operator 108 in the Z direction. In some embodiments, the display unit 206 can also be moveable in other DOFs (not shown).

For example, in some embodiments, the control module 170 can determine the distance 304, or another geometric parameter as described above, based on an estimated interpupillary distance in captured images and a function (e.g., a linear function or a non-linear function) or a lookup table or other construct relating a ratio between the estimated interpupillary distance and a reference interpupillary distance to the distance 304, or the other geometric parameter, the size of an iris or other immutable feature of the eyes 302 in captured images, parallax between pupils detected in stereo images, a distance between the eyes 302 or other portion(s) of the head of the operator 108 and the lenses or other portion(s) of the display system 200 in side view images of a head of the operator 108, or time-of-flight sensor data corresponding to the eyes 302 or other portion(s) of the head of the operator 108, as described above in conjunction with FIGS. 6-9. Then, the control module 170 can determine an inward-outward movement of the display unit 206 in the degree of freedom 222, described above in conjunction with FIG. 2, such that the lenses 223 or other portion(s) of the display system 200 are moved from the determined distance 304, or the other geometric parameter, relative to the eyes 302 of the operator 108 to the target distance, or another target parameter, relative to the eyes 302 of the operator 108. Thereafter, the control module 170 can issue one or more commands, directly or indirectly, to one or more actuators 312 that cause the display unit 206 to linearly translate in the linear degree of freedom 222, described above in conjunction with FIG. 2, such that the display unit 206 coupled to the second arm portion moves according to the determined movement.

In some embodiments, the control module 170 can further command another actuator in the actuator system to drive the repositionable structure system in accordance with a second commanded motion, and move the headrest 242 relative to the display unit 206 by a same magnitude and in an opposite direction to the movement the headrest 242 would have experienced with the first commanded motion without the second commanded motion (also referred to herein as a “complementary motion”); this technique can maintain the headrest 242 in one or more degrees of freedom, such as a position of the headrest 242 in one or more dimensions and/or an orientation of the headrest 242 about one or more axes. Maintaining the headrest 242 in one or more degrees of freedom can reduce motion of a head position of the operator 108, when the head is in contact with the headrest 242. In such cases, the headrest 242 can remain substantially stationary relative to the head of the operator 108 and/or relative to a common frame of reference such as a world frame, while other joints of the repositionable structure are moved to move the display unit 206. For example, in some embodiments, the display system 200 includes a single repositionable structure having a number of degrees of freedom that can be used to move the display unit 206 and an additional degree of freedom, shown as degree of freedom 320, that can be used to move the headrest 242 relative to the display unit 206. In other embodiments, the display unit 206 can be mounted or otherwise physically coupled to a first repositionable structure of the repositionable structure system, and the headrest 242 can be mounted or otherwise physically coupled to a second repositionable structure of the repositionable structure that moves the headrest 242 along the degree of freedom 320. The second repositionable structure can physically extend from the first repositionable structure, or be physically separate from the first repositionable structure. An example complementary motion 308 of the headrest 242 by a same magnitude and in an opposite direction to the example movement 306, which causes the headrest 242 to move farther away from the display unit 206, is shown in FIG. 3. As another example, the complementary motion can cause the headrest 242 to move closer to the display unit 206 when the motion of the display unit 206 is moved toward the operator 108. As a result of the motion of the display unit 206 and the complementary motion of the headrest 242, the distance 304, or another geometric parameter as described above, can be changed while the headrest 242 remains stationary relative to the operator 108. More generally, in some embodiments, a repositionable structure system can be moved based on a commanded motion to maintain the headrest 242 in at least one degree of freedom in a common frame of reference when the display unit 206 and a base for the headrest 242 are moved in the common reference frame. The control module 170 can issue one or more commands, directly or indirectly, to one or more actuators (e.g., actuator 316) that cause the headrest 242 to move according to the complementary movement.

In some embodiments, the actuator 316 is a linear actuator that is configured to move/adjust the position of headrest 242 along the Z-axis to actively position the head of the operator 108 in a direction parallel to an optical axis of the lenses 223. In operation, the actuator 316 can be controlled by any technically feasible control system, such as the control module 170, and/or operator input to move the headrest 242. In particular, in some embodiments, the control system and/or operator input devices can communicate, directly or indirectly, with an encoder (not shown) included in the actuator 316 to cause a motor to rotate a ball screw (not shown). As the ball screw rotates, a ball screw nut (not shown) that is coupled to a sled 330 moves along the Z-axis on a rail (not shown). The sled 330 is, in turn, coupled to a shaft 332 of the headrest 242 and slidably connected to the rail. Thus, the headrest 242 is moved along the Z-axis. Although described herein primarily with respect to a ball screw linear actuator, other mechanisms can be employed to adjust/move a headrest of a display unit in accordance with the present disclosure. For example, other electromechanical, or a mechanical, hydraulic, pneumatic, or piezoelectric actuator can be employed to move an adjustable headrest of a display unit in accordance with this disclosure. As examples, a geared linear actuator or a kinematic mechanism/linkage could be employed to move the headrest 242. Additional examples of moveable display systems are described in U.S. Provisional Patent Application No. 63/270,418 having attorney docket number P06424-US-PRV, filed Oct. 21, 2021, and entitled “Adjustable Headrest for a Display Unit,” which is incorporated by reference herein.

In some embodiments that include lenses or display screens, the lenses (e.g., lenses 223) or the display screens can move separately from the rest of the display unit 206. For example, the lenses 223 or the display screens could be coupled to a track or cart mechanism that permits the lenses 223 or the display screens to be moved in an inward-outward direction relative to the display unit 206. As used herein, the inward-outward direction is a direction parallel to a direction of view of the operator 108. An example movement 310 of the lenses 223 in a direction that increases the distance 304 is shown in FIG. 3. As shown, the lenses 223 can generally be moved in a DOF 324 closer or farther away from the eyes 302 of the operator 108 in the Z direction. Similar to the headrest 242, in some embodiments, each lens 223 is coupled to a corresponding sled 334 that slides along rails, and the lens 223 can be moved in the Z direction by commanding a corresponding actuator 314 to cause a motor to rotate a ball screw that moves a ball screw nut that is coupled to the sled 334 that is coupled to the lens 223. In other embodiments, alternative mechanisms can be employed to adjust/move lenses or a display screen of a display unit, relative to other parts of the display unit (e.g., a housing of the display unit), in accordance with the present disclosure. For example, other electromechanical, or a mechanical, hydraulic, pneumatic, or piezoelectric actuators can be employed to move the lenses or a display screen in accordance with this disclosure. In some examples, the control module 170 can determine an inward-outward movement of the lenses 223 (or a display screen), independently of other part(s) of the display unit 206, from the determined distance 304 relative to the eyes 302 of the operator 108 to the target distance relative to the eyes 302 of the operator 108. The control module 170 can then issue commands, directly or indirectly, to the actuators 314 coupled to the lenses to cause the lenses 223 (or a display screen) to move according to the determined movement

In some examples, the headrest 242 can be moved in the inward-outward direction relative to the display unit 206 so that the head of the operator 108 that is in contact with the headrest 242 is moved closer or farther away relative to the lenses 223, or other portion(s) of the display system 200. In some examples, the control module 170 can further determine an inward-outward movement of the lenses 223, or other portion(s) of the display system 200, that causes the head of the operator 108 that is in contact with the headrest 242 to move relative to the lenses 223 such that the eye-to-lens distance, or another geometric parameter, changes from the determined distance or other geometric parameter to the target distance relative to the eyes 302 of the operator 108 or another target parameter. Then, the control module 170 can issue commands to a controller for one or more joints of a repositionable structure to which the headrest 242 is mounted or otherwise physically coupled to cause movement of the headrest 242 according to the determined movement. For example, based on the determined movement, the control module 170 can issue one or more commands, directly or indirectly, to the actuator 316, as described above in conjunction with the complementary motion of the headrest 242, to move the headrest 242 to move the eyes 302 of the operator 108 to the target distance relative to the lenses 223, or according to another target parameter. More generally, in some embodiments, a repositionable structure to which the headrest 242 is physically coupled can be moved based on a commanded motion to maintain the headrest 242 in at least one degree of freedom in a common frame of reference when the display unit 206 is moved in the common reference frame. Although described herein primarily with respect to moving the headrest 242 in the inward-outward direction, in other embodiments the headrest 242 can also be moved in other directions and/or rotations, such as about the yaw axis 230 based on a motion of the eyes 302 of the operator 108.

In some embodiments with a head input mode, the target parameter does not differ when the control system is in the head input mode and when the control system is not in the head input mode. In some embodiments with a head input mode, commanded motion determined for the repositionable structure system to move (e.g., to move a headrest, to move the entirety of the display unit 206, to move the lenses 223 or other portion(s) of the display unit 206) is based on a second target parameter different from the target parameter used when not in the head input mode. This difference can be temporary, and reduce with the passage of time in the head input mode, or remain partially or entirely while in the head input mode.

The head input mode can be entered in any technically feasible manner. In some embodiments, the head input mode can be entered in response to a button being pressed, hand input sensed by hand-input sensors (e.g., the hand-input sensors 240a-b) meeting particular criteria, etc. In some embodiments, when the head input mode is entered, the repositionable structure system can be commanded to reposition the headrest 242 relative to the display unit 206 by moving the display unit 206, the headrest 242, or both the display unit 206 and the headrest 242, so that the headrest 242 moves away from the display unit 206. For example, the headrest may be repositioned to an extended position relative to the display unit 206. For example, the headrest 242 may be extended to a furthest extension defined by the system. The headrest 242 extension can then be maintained while in the head input mode, or reduced gradually or in a stepwise-manner in response to a passage of time, exit from head input mode, or some other trigger event. As an example, the headrest 242 may be extended at an increased distance (e.g., a maximum permissible distance from the display unit 206) based on a value defined independently of the target parameters. The value can then be decreased, also independently of the target parameters. In some embodiments, when the head input mode is entered, the system can use a second target parameter different from a non-head-input-mode (“ordinary”) target parameter. For example, the second target parameter could correspond to the larger extension (e.g., a maximum permissible extension or some other defined extension) of the headrest 242 relative to the display unit 206. As a particular example, the increased extension could correspond to a separation distance of 25 mm between the headrest 242 and the display unit 206, and the non-head-input ordinary target distance could be 15 to 20 mm. The system can then define a sequence of further target parameters corresponding to smaller extensions of the headrest 242 relative to the display unit 206, and ending with a target parameter unassociated with the head input mode (which may be equal to the ordinary target parameter). The sequence of target parameters can reduce the target parameter from the second target parameter to the ordinary target parameter over a number of time steps or by following a ramping or any other monotonic time function. Such a reduction of the target distance or other target parameter is also referred to herein as “ratcheting” because the target distance or other target parameter is effectively ratcheted from the increased distance to the ordinary target distance. For example, the system can determine, over a period of time, a sequence of further target parameters, each further target parameter being between the second target parameter and the ordinary target parameter and being closer to the ordinary target parameter than the immediately previous further target parameter in the sequence. The control system can then command, during that period of time or shortly after that period of time, the actuator system to drive the repositionable structure system based on further commanded motions determined based on the further target parameter values, such that the headrest 242 can be repositioned accordingly. The change in the extension amount of the headrest, or the target parameter values, can be in response to a trigger event such as passage of a period of time after entry into the head input mode, a passage of a defined duration of time after the actuator system has moved the second repositionable structure based on the second commanded motion, a magnitude of a velocity and/or acceleration of the display unit 206 decreasing below a threshold magnitude of velocity and/or acceleration, or an exit from the head input mode.

In some embodiments, after the head input mode is entered, another target parameter is used temporarily or throughout the entirety of the head input mode, to change the behavior of the system. For example, the another target parameter may correspond to an increased separation distance (e.g., a maximum acceptable or other larger distance) compared to the separation distance associated with the non-head-input (“original”) target parameter. Commanded motion is determined based on this another target parameter, and the actuator system is commanded to move the repositionable structure system accordingly.

Where the behavior is made temporary, the control system can determine a sequence of target parameters that corresponds to reducing the separation distance back down to a non-head-input target distance, as described above. In other embodiments, the target parameter can be reset to a non-head-input target parameter in one step, such that the increased distance is reset to the ordinary target distance in a single step. It should be understood that the target parameter can be changed regardless of any determination of current geometric parameters or of any sensor signals (e.g., can be changed just in response to the entry into the head input mode). Temporarily using the second target parameter to increase the separation distance can help prevent a head of the operator 108 from inadvertently contacting parts of the display unit 206. When the display system 200 is configured to receive head input (e.g., forces) through the headrest 242 and not other parts of the display unit 206 in the head input mode, if the head of the operator contacts a part of the display unit 206 other than the headrest 242, then some of the input (e.g., force, torque) provided by the head can be transmitted through that part of the display unit 206 instead of the headrest 242. In such cases, the head input would not be accurately sensed, and the system response can become erroneous, unexpected, or otherwise aberrant.

In some embodiments, the target parameter is changed to one corresponding to an increased distance for a predefined duration of time (e.g., 30 seconds to a few minutes), and passage of the predefined duration of time is the trigger event that causes the sequence of further target parameters to reduce the corresponding separation distances back down to the ordinary target distance over a period of time (e.g., 10 to 30 seconds). In some embodiments, a magnitude of the velocity of the display unit 206, which follows the motion of the head of the operator, decreasing below a threshold magnitude of velocity (e.g., 0.5 rads/s in every axis) and/or a magnitude of the acceleration of the display unit 206 decreasing below a threshold magnitude of acceleration is the trigger event that causes sequence of target parameters corresponding to target distances that to reduce back down to the ordinary target distance over a period of time (e.g., 2 to 5 seconds). In such cases, when the velocity exceeds the threshold magnitude of velocity and/or the acceleration exceeds the threshold magnitude of acceleration again, the reduction can be paused until the magnitude of the velocity decreases below the threshold magnitude of velocity and/or the magnitude of the acceleration decreases below the threshold magnitude of acceleration. In yet further embodiments, exiting of the head input mode or another mode is the trigger event that causes the sequence of further target parameters corresponding to target distances no reduce back down to the ordinary target distance or other target parameter over a period of time (e.g., 2 to 5 seconds). In such cases, the likelihood that the user contacts the display unit 206 (e.g., the face of the user is kept clear of the display unit) can be reduced while the control system of the computer-assisted device is in the head input mode. In the examples described in conjunction with FIG. 3, inverse kinematics can be used to compute joint velocities or positions for joints associated with the display unit 206, and/or the repositionable structure system to which the display unit 206, the headrest 242, and/or the lenses 223 are physically coupled, that will move the display unit 206, the headrest 242, and/or the lenses 223 toward achieving the commanded motions.

In some embodiments, various parameters described herein, such as the target parameters, the periods of time, the threshold magnitudes of velocity and/or acceleration, the scaling factor, etc., can be determined based on one or more of a type of the lenses, a type of the display unit, a type of the repositionable structure system, operator preference, a type of a procedure being performed at the worksite, a calibration procedure, among other things.

FIG. 8 illustrates a simplified diagram of a method 800 for adjusting a geometric relationship (a distance is used in parts of the example of FIG. 8) between the one or more portions of the head of an operator and one or more portions of a display system, according to various embodiments. One or more of the processes 802-816 of method 800 may be implemented, at least in part, in the form of executable code stored on non-transitory, tangible, machine readable media that when run by one or more processors (e.g., the processor 150 in control system 140) may cause the one or more processors to perform one or more of the processes 802-816. In some embodiments, method 800 may be performed by one or more modules, such as control module 170. In some embodiments, method 800 may include additional processes, which are not shown. In some embodiments, one or more of the processes 802-816 may be performed, at least in part, by one or more of the modules of control system 140.

As shown, the method 800 begins at process 802, where sensor data associated with the head of an operator (e.g., operator 108) is received. Any technically feasible sensor data can be received, such as image and/or time-of-flight data from the cameras 404, 502, 504, 506, 508, 602, 602, 704 and/or the time-of-flight sensor 702 in one of the configurations described above in conjunction with FIGS. 6-9.

At process 804, a geometric parameter of that is representative of a geometric relationship of the eye(s) of an operator relative to the image(s) displayed by a display unit is determined based on the sensor data. Examples of the geometric parameter are described above in conjunction with FIG. 3. The geometric parameter can be determined in any technically feasible manner, such as based on an estimated interpupillary distance in captured images and a function (e.g., a linear function or a non-linear function) or a lookup table or other construct relating a ratio between the estimated interpupillary distance and a reference interpupillary distance to an eye-to-lenses distance, the size of an iris or other immutable feature of the eyes in captured images, parallax between pupils detected in stereo images, scaling a distance between, or the locations of, the eyes or other portion(s) of the head of the operator and the portion(s) of the display system in side view images of a head of the operator, or time-of-flight sensor data, as described above in conjunction with FIGS. 5-9. If separate geometric parameters are determined for both eyes of an operator, those geometric parameters can be averaged in some embodiments. In other embodiments, a single geometric parameter of one eye or other portion of the head of the operator relative to portion(s) of the display system can be determined and used as the geometric parameter at process 804.

At process 806, a commanded motion of the display unit, a repositionable structure system coupled to the display unit, a headrest (e.g., headrest 242), or the lenses is determined based on the geometric parameter determined at process 804 and a target parameter. The commanded motion is a motion in a direction parallel to a direction of view of the operator (e.g., the direction Z in FIG. 3) that moves the display unit, the repositionable structure system, the headrest, or the lenses from a current position so that the one or more portions of the display system are the target parameter (e.g., a target distance) relative to the eyes of the operator, i.e., the geometric parameter of the eyes relative to the one or more portions of the display system is equal to the target parameter. In other embodiments, the commanded motion can include a motion of a combination of the display unit, the repositionable structure system, the headrest, and/or the lenses in a direction parallel to a direction of view of the operator. When a commanded motion is determined for moving the display unit, a complementary motion of the headrest can also be determined. The complementary motion is described above in conjunction with FIG. 3. By moving the display unit according to the commanded motion and the headrest according to the complementary motion, the position and/or orientation of the headrest can be maintained in at least one dimension. Doing so can help reduce the amount of head motion required of the head of the operator, or leave substantially unchanged the head position of an operator whose head is in contact with the headrest.

A repositionable structure system is physically coupled to the display unit, the headrest, and/or the lenses. At process 808, a repositionable structure system is actuated based on the commanded motion. In some embodiments, a repositionable structure system to which the display unit, the repositionable structure system, the headrest, or the lenses is mounted or otherwise coupled can be actuated by transmitting signals, such as voltages, currents, pulse-width modulations, etc. to one or more actuators (e.g., the actuators 312, 314, and/or 316 described above in conjunction with FIG. 3) of an actuator system configured to move the repositionable structure system. Actuators of the actuator system may be located in the repositionable structure, or be at least partially separate from the repositionable structure system with motive forces and torques transmitted to the repositionable structure system through one or more transmission components. In some embodiments, when a first repositionable structure to which the display unit is coupled is actuated based on a commanded motion for moving the display unit, a second repositionable structure to which the headrest is coupled can be moved contemporaneously or within a period of time of the commanded motion, based on a second commanded motion. The second commanded motion can be determined to maintain the headrest in at least one degree of freedom in a common frame of reference such as a world frame, when the display unit is moved (and not maintained in the at least one degree of freedom in the common reference frame).

At process 810, when an adjustment by the operator to the position of the display unit or the repositionable structure system is detected, then at process 812, the target parameter is reset based on the position of the display unit or the repositionable structure system position after the adjustment. Although processes 810-812 are shown as following process 808, in some embodiments, the target parameter can be reset based on an adjustment to the position of the display unit or the repositionable structure system by the operator at any time.

Alternatively, when no adjustment by the operator to the position of the display unit or the repositionable structure system is detected at process 814, and after the target parameter is reset at process 816, the method 800 returns to process 802, where additional sensor data associated with one or both eyes of the operator is received.

FIG. 9 illustrates a simplified diagram of a method for adjusting a repositionable structure system in response to entry into a mode in which a display unit is commanded to move based on head force and/or torque measurements, according to various embodiments. One or more of the processes 902-908 of method 900 may be implemented, at least in part, in the form of executable code stored on non-transitory, tangible, machine readable media that when run by one or more processors (e.g., the processor 150 in control system 140) may cause the one or more processors to perform one or more of the processes 902-908. In some embodiments, method 900 may be performed by one or more modules, such as control module 170. In some embodiments, method 900 may include additional processes, which are not shown. In some embodiments, one or more of the processes 902-908 may be performed, at least in part, by one or more of the modules of control system 140.

As shown, the method 900 begins at process 902, where the control system enters a head input mode in which the position and/or orientation of a display unit (e.g., display unit 206) is driven based on head applied force, and/or head applied torque, and/or head motion (e.g., change in position, velocity, acceleration). The head input may be detected as one or more measurements obtained with a sensor. The head input mode is entered in response to operator (e.g., operator 108) input. For example, the mode could be a head input mode, described above in conjunction with FIGS. 1-2. The head input mode can be entered in any technically feasible manner, such as in response to a button being pressed by the operator, hand input sensed by hand-input sensors (e.g., the hand-input sensors 240a-b) meeting particular criteria, etc.

At process 904, a repositionable structure to which the display unit or a headrest (e.g., headrest 242) is mounted or otherwise physically coupled is actuated based on a first target parameter of the one or more portions (e.g., eyes 302) of the head of the operator relative to one or more portions (e.g., lenses 223) of the display system. In some embodiments, the first target parameter is a maximum acceptable separation distance, such as 25 mm. In some embodiments, the method 800, described above in conjunction with FIG. 8, can be performed to move the display system or headrest the first target parameter relative to the eyes of an operator.

At process 906, in response to a trigger event, the repositionable structure to which the display unit or the headrest is coupled is actuated based on a sequence of target parameters spanning from the first target parameter to a second target parameter. In some embodiments, the trigger event is the passage of a defined duration of time after the control system of the computer-assisted device has entered the mode in which the position of the display unit is driven based on head force and/or torque measurements. For example, the duration of time can be anywhere between 30 seconds and a few minutes. In some embodiments, the trigger event is a magnitude of a velocity of the display unit decreasing to less than a threshold magnitude of velocity and/or a magnitude of an acceleration of the display unit decreasing to less than a threshold magnitude of acceleration. In such cases, when the velocity exceeds the threshold magnitude of velocity and/or the acceleration exceeds the threshold magnitude of acceleration again, the reduction can be paused until the magnitude of the velocity decreases below the threshold magnitude of velocity and/or the magnitude of the acceleration decreases below the threshold magnitude of acceleration. For example, the threshold magnitude of velocity could be 0.5 rads/s in every axis. In some embodiments, the trigger event is the exiting of the mode in which the position of the display unit is driven based on head force and/or torque measurements.

In some embodiments, the second target parameter, to which the target parameter is reduced, is an ordinary target parameter, such as a 15-20 mm separation distance. In some embodiments, the target parameter is ratcheted by reducing the target parameter from the first target parameter to the second target parameter over a period of time (e.g., seconds) through a number of time steps or by following a ramping or any other monotonic time function. In such cases, further target parameters between the second target parameter and the target parameter can be determined over the period of time, and the repositionable structure to which the display unit is coupled can be actuated according to commands that are generated based on the further target parameters. In other embodiments, the target distance, or another target parameter, can be reduced directly from the maximum acceptable distance, or other target parameter, to the ordinary target distance, or ordinary target parameter, in a single step (i.e., ratcheting can be omitted)

As described, in various ones of the disclosed embodiments, a geometric parameter that is representative of a geometric relationship of the eye(s) of an operator relative to the image(s) displayed by a computer-assisted device is determined from sensor measurements. In some embodiments, the geometric parameter can be determined by detecting pupils in images captured by cameras, estimating a distance between the pupils, and computing a distance based on the estimated distance, a reference distance between the pupils, and a function (e.g., a linear function or a non-linear function) or a lookup table or other construct. Such a function or lookup table or other construct can be obtained, for example, through extrapolation or interpolation of data, including through linear regression. In some embodiments, the geometric parameter can be determined by detecting an iris or other immutable feature in an image captured by a camera, measuring a size of the iris or other immutable feature, and computing the geometric parameter based on the measured size and a reference size of the iris or other immutable feature. In some embodiments, the geometric parameter can be determined based on parallax between pupils that are detected in stereo images captured by pairs of cameras. In some embodiments, the geometric parameter can be determined by detecting eyes (or other portion(s) of the head) of the operator in images captured by a camera or set of cameras on the sides of an operator, and scaling distances or relative locations between the eyes (or other portion(s) of the head) and portion(s) of the computer-assisted device in the images. In some embodiments, the distance can be determined by identifying eyes or other portion(s) of the head of an operator in images captured by one or more cameras, and computing distances based on time-of-flight sensor data corresponding to the eyes or other portion(s) of the head.

The one or more portions (e.g., lenses) of the display unit are repositioned from the determined geometric parameter based on a target parameter relative to the one or more portions of the head of the operator by moving the display unit, a repositionable structure system physically coupled to the display unit, the lenses relative to the display unit, or a headrest relative to the display unit. When the display unit is moved, the headrest can be moved according to a complementary motion so that a head of the operator that is in contact with the headrest can remain substantially stationary.

The disclosed techniques can automatically reposition one or more portions of a computer-assisted device relative to one or more portions of the head of an operator. Such a repositioning can permit the operator to see an entire image being displayed via a display unit of the computer-assisted device and/or to see a properly fused image that combines images seen through different lenses, when the display unit includes lenses. Further, operator eye fatigue can be avoided or reduced. In addition, when a head input mode is entered, the headrest or one or more portions of the computer-assisted device can be repositioned away from the operator to help prevent the head of the operator from inadvertently contacting the display unit.

Some examples of control systems, such as control system 140 may include non-transitory, tangible, machine readable media that include executable code that when run by one or more processors (e.g., processor 150) may cause the one or more processors to perform the processes of methods 800, 900, and/or 1000 and/or the processes of FIGS. 8, 9, and/or 10. Some common forms of machine readable media that may include the processes of methods 800, 900, and/or 1000 and/or the processes of FIGS. 8, 9, and/or 10 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

1. A computer-assisted device comprising:
a repositionable structure system configured to physically couple to a display unit, the display unit configured to display images viewable by an operator;
an actuator system physically coupled to the repositionable structure system, the actuator system drivable to move the repositionable structure system;
a sensor system configured to capture sensor data associated with a portion of a head of the operator; and
a control system communicably coupled to the actuator system and the sensor system,
wherein the control system is configured to:
determine, based on the sensor data, a geometric parameter of the portion of the head relative to a portion of the computer-assisted device selected from the group consisting of: portions of the display unit and portions of the repositionable structure system, the geometric parameter being representative of a geometric relationship of at least one eye of the operator relative to an image displayed by the display unit,
determine a commanded motion based on the geometric parameter and a target parameter, and
command the actuator system to move the repositionable structure system based on the commanded motion, wherein the commanded moves at least one of a lens of the display unit relative to a housing of the display unit or a headrest of the display unit relative to the display unit.

2. (canceled)

3. The computer-assisted device of claim 1, wherein:
the geometric parameter and the target parameter differ by a first difference;
moving the repositionable structure system based on the commanded motion would cause the at least one eye to have an updated geometric relationship relative to the image, the updated geometric relationship representable by an updated geometric parameter;
the updated geometric parameter and the target parameter differ by a second difference; and
the second difference is smaller than the first difference.

4. The computer-assisted device of claim 1, wherein the portion of the computer-assisted device comprises the lens, the lens being positioned between a location of the image and an expected location of the at least one eye.

5. The computer-assisted device of claim 1, wherein the geometric relationship is an optical distance between the at least one eye and the image.

6-8. (canceled)

9. The computer-assisted device of claim 1, wherein:
the computer-assisted device further comprises a sensor configured to detect kinematic information about the repositionable structure system; and
to determine the geometric parameter, the control system is configured to use the kinematic information to determine a physical configuration of the repositionable structure system.

10. The computer-assisted device of claim 1, wherein the at least one eye comprises a first eye and a second eye, and wherein determining the geometric parameter comprises:
identifying pupils of the first and second eyes based on the sensor data;
determining a sensed distance between the pupils; and
determining the geometric parameter based on the sensed distance between the pupils and a reference distance between the pupils.

11. The computer-assisted device of claim 1, wherein determining the geometric parameter comprises:
determining a size of a component of the at least one eye based on the sensor data; and
determining the geometric parameter based on the size of the component and a reference size of the component.

12. The computer-assisted device of claim 1, wherein determining the geometric parameter comprises:
determining a separation distance between the portion of the head and a portion of the computer-assisted device based on the sensor data; and
scaling the separation distance using a scaling factor.

13. The computer-assisted device of claim 1, further comprising:
the headrest configured to contact a forehead of the operator;
wherein the repositionable structure system comprises a first repositionable structure configured to support the display unit and a second repositionable structure coupled to the headrest and drivable to move the headrest relative to the display unit,
wherein the actuator system moving the repositionable structure system based on the commanded motion moves the first repositionable structure such that the display unit moves in a common frame of reference, and
wherein the control system is further configured to:
determine a second commanded motion based on the commanded motion, wherein driving the second repositionable structure in accordance with the second commanded motion while moving the first repositionable structure based on the commanded motion maintains the headrest in at least one degree of freedom in the common frame of reference, and
drive the second repositionable structure in accordance with the second commanded motion.

14-16. (canceled)

17. The computer-assisted device of claim 1, further comprising:
the headrest, wherein the repositionable structure system comprises a first repositionable structure configured to support the display unit and a second repositionable structure physically coupled to the headrest and drivable to move the headrest relative to the display unit; wherein
the control system is further configured to:
enter a head input mode in which the control system commands the first repositionable structure to move the display unit based on at least one head input selected from the group consisting of: a head motion, an applied force of the head, and an applied torque of the head, and
in response to the control system entering the head input mode, command the second repositionable structure to move the headrest away from the display unit.

18. (canceled)

19. The computer-assisted device of claim 1, further comprising:
the headrest, wherein the repositionable structure system comprises a first repositionable structure configured to support the display unit and a second repositionable structure coupled to the headrest and drivable to move the headrest relative to the display unit; wherein
the control system is further configured to:
enter a head input mode in which the control system commands the first repositionable structure to move the display unit based on at least one head input selected from the group consisting of: a head motion, an applied force of the head, and an applied torque of the head, and
in response to the control system entering the head input mode:
determine a second commanded motion based on a second target parameter different from the target parameter, and
command the actuator system to move the second repositionable structure based on the second commanded motion.

20. (canceled)

21. The computer-assisted device of claim 19, wherein the control system is further configured to, after the display unit or the repositionable structure system has entered the head input mode and in response to a trigger event:
determine, over a period of time, a sequence of further target parameters, each further target parameter being between the second target parameter and the target parameter and being closer to the target parameter than an immediately previous further target parameter in the sequence.
determine, over the period of time, a sequence of further commanded motions based on the sequence of further target parameters; and
command, over the period of time, the actuator system to move the second repositionable structure based on the sequence of further commanded motions.

22. (canceled)

23. The computer-assisted device of claim 1, wherein the control system is further configured to, in response to a manual repositioning of the display unit, set the target parameter based on:
a distance between the portion of the head and the portion of the computer-assisted device at a completion of the manual repositioning; or
a location of the portion of the head relative to the portion of the computer-assisted device at the completion of the manual repositioning.

24. The computer-assisted device of claim 1, wherein the display unit further comprises an optical element, wherein the sensor system comprises a sensor disposed behind the optical element, in a direction away from the operator.

25. The computer-assisted device of claim 1, wherein the target parameter is determined based on at least one input selected from the group consisting of:
a physical configuration of the display unit;
a type of an optical component of the display unit; and
a display related characteristic of the display unit.

26. A method for controlling a computer-assisted device comprising a repositionable structure system configured to physically couple to a display unit, the method comprising:
determining, based on sensor data, a geometric parameter of a portion of a head of an operator relative to a portion of the computer-assisted device selected from the group consisting of: portions of the display unit and portions of the repositionable structure system, the geometric parameter being representative of a geometric relationship of at least one eye of the operator relative to an image displayed by the display unit;
determining a commanded motion based on the geometric parameter and a target parameter; and
commanding an actuator system to move the repositionable structure system based on the commanded motion, wherein the commanded moves at least one of a lens of the display unit relative to a housing of the display unit or a headrest of the display unit relative to the display unit.

27-28. (canceled)

29. The method of claim 26, wherein the geometric relationship is an optical distance between the at least one eye and the image.

30. (canceled)

31. The method of claim 26, wherein the at least one eye comprises a first eye and a second eye, and wherein determining the geometric parameter comprises:
identifying pupils of the first and second eyes based on the sensor data;
determining a sensed distance between the pupils; and
determining the geometric parameter based on the sensed distance between the pupils and a reference distance between the pupils.

32. The method of claim 26, wherein determining the geometric parameter comprises:
determining a size of a component of the at least one eye based on the sensor data; and
determining the geometric parameter based on the size of the component and a reference size of the component.

33. (canceled)

34. The method of claim 26, wherein the repositionable structure system comprises a first repositionable structure configured to support the display unit and a second repositionable structure coupled to the headrest and drivable to move the headrest relative to the display unit, wherein the actuator system moving the repositionable structure system based on the commanded motion moves the first repositionable structure such that the display unit moves in a common frame of reference, the method further comprising:
determining a second commanded motion based on the commanded motion, wherein driving the second repositionable structure in accordance with the second commanded motion while moving the first repositionable structure based on the commanded motion maintains the headrest in at least one degree of freedom in the common frame of reference; and
driving the second repositionable structure in accordance with the second commanded motion.

35-40. (canceled)

41. The method of claim 26, further comprising in response to a manual repositioning of the display unit, setting the target parameter based on:
a distance between the portion of the head and the portion of the computer-assisted device; or
a location of the portion of the head relative to the portion of the computer-assisted device.

42. (canceled)

43. One or more non-transitory machine-readable media comprising a plurality of machine-readable instructions which, when executed by one or more processors associated with a computer-assisted device comprising a repositionable structure system configured to physically couple to a display unit, are adapted to cause the one or more processors to perform a method comprising:
determining, based on sensor data, a geometric parameter of a portion of a head of an operator relative to a portion of the computer-assisted device selected from the group consisting of: portions of the display unit and portions of the repositionable structure system, the geometric parameter being representative of a geometric relationship of at least one eye of the operator relative to an image displayed by the display unit;
determining a commanded motion based on the geometric parameter and a target parameter; and
commanding an actuator system to move the repositionable structure system based on the commanded motion, wherein the commanded moves at least one of a lens of the display unit relative to a housing of the display unit or a headrest of the display unit relative to the display unit.
